(19) United States
(12) Patent Application Publication
(10) Pub. No.: US 2018/0177482 A1
(43) Pub. Date: Jun. 28, 2018

(54) BIOLOGICAL SOUND MEASUREMENT APPARATUS

(71) Applicant: OMRON HEALTHCARE CO., LTD., Muko-shi (JP)

(72) Inventors: Kenji HASHINO, Kyoto (JP); Makoto TABATA, Kyoto (JP); Nobuki YAKURA, Kyoto (JP); Takayuki SHIINA, Kyoto (JP)

(21) Appl. No.: 15/849,832

(22) Filed: Dec. 21, 2017

(30) Foreign Application Priority Data
Dec. 28, 2016 (JP) 2016-255759

(51) Int. Cl. A61B 7/00; A61B 5/00

(52) U.S. Cl. CPC A61B 7/003; A61B 5/683; A61B 7/04; A61B 5/6822; A61B 5/7221; A61B 5/7285

(57) ABSTRACT

A biological sound measurement apparatus includes a housing that has an opening and defines a housing space in which a sound detector is housed, and a housing cover that covers the housing. An outer wall surface of the housing includes a first wall surface that protrudes farthest toward a body surface in a contact state and includes the opening, a step wall surface that is closer to the sound detector than the first wall surface, a first side wall surface that connects a level difference between the step wall surface and the first wall surface, and a second side wall surface that is bent relative to the step wall surface. The housing cover includes a pressure receiving portion that faces the first wall surface, and a first corresponding wall portion, a second corresponding wall portion, and a third corresponding wall portion that are in close contact with the first side wall surface, the step wall surface, and the second side wall surface.

### CROSS REFERENCE TO RELATED APPLICATIONS

This application claims the benefit of priority to Japanese Patent Application No. 2016-255759 filed on Dec. 28, 2016. The entire contents of this application are hereby incorporated herein by reference.

### BACKGROUND OF THE INVENTION

### 1. Field of the Invention

The present invention relates to a biological sound measurement apparatus that is used by being brought into contact with a body surface of a biological body.

### 2. Description of the Related Art

Apparatuses are known that use a sound detector such as a microphone to pick up a biological sound such as a cardiac sound or a respiratory sound (e.g., wheezing) as an electrical signal. As an example of this type of apparatus, JP H7-16497U discloses a contact-type of indirectly transmitted vibration pickup microphone that is fixed to the throat of a user and measures sound produced by the vocal cords.

In the microphone disclosed in JP H7-16497U, a pressure fluctuation space is formed by an upper lid, a main body, and a vibration damping gel member that is provided inside the main body, and a capacitor microphone is disposed in the pressure fluctuation space. The upper lid that forms the pressure fluctuation space is shaped as a bottomed tube and functions as a vibration receiving piece, and when this vibration receiving piece is brought into contact with a throat and vibrates in accordance with a biological sound made by a biological body, the pressure in the pressure fluctuation space changes, and the biological sound is measured by the microphone.

JP 3080779U discloses a body-contact type of voltage-type microphone. In this microphone, a closed space is formed by combining a front lid and a rear lid, and a vibration detection member that is defined by a ceramic piece is arranged in the closed space. The front lid is shaped as a bottomed tube and is fitted to an outer peripheral portion of the rear lid. In this configuration, the front edge portion of the front lid is brought into contact with the user's skin, and a biological sound is transmitted from the front edge portion to the vibration detection member via a spring in the closed space.

JP 2000-60845A discloses a biological sound detection apparatus that is used when mounted to the skin of a biological body. In this biological sound detection apparatus, an opening is formed in a protruding portion of a housing that has a protruding shape and forms a housing space in which a microphone is housed. In this configuration, the opening is blocked by a flexible resin film that has a bottom tube shape and is fitted around the protruding portion of the housing.

When a biological sound measurement apparatus performs biological sound measurement that is necessary for medical examination of a biological body, a certain biological sound measurement precision is required of the biological sound measurement apparatus. In order to increase the biological sound measurement precision, it is effective to increase the internal pressure of the pressure fluctuation space in which the microphone is housed.

In order to increase the internal pressure of the pressure fluctuation space, it is conceivable to increase the air pressure in the environment in which the biological sound measurement apparatus is assembled, but this method requires equipment for controlling the air pressure, and thus the manufacturing cost of the apparatus rises. Also, even if the internal pressure of the pressure fluctuation space is high in the manufacturing stage, if the internal pressure is not maintained over a long period of time, it is not possible to maintain the measurement precision over a long period of time.

The apparatuses disclosed in JP H7-16497U, JP 3080779U and JP 2000-60845A are apparatuses in which a pressure fluctuation space is blocked by a member that has bottomed tube shape and is fitted to a housing or the like. However, with the bottomed tube-shaped members disclosed in JP H7-16497U, JP 3080779U and JP 2000-60845A, the contact region is small between the bottomed tube-shaped member and the portion to which it is fitted, and therefore there is a possibility that the internal pressure of the pressure fluctuation space escapes through this contact region, and the detection precision decreases.

Also, in order to prevent the escape of the internal pressure of the pressure fluctuation space in the apparatuses disclosed in JP H7-16497U, JP 3080779U and JP 2000-60845A, the width of the housing that forms the pressure fluctuation space needs to be increased in the direction in which the housing is pressed against the biological body, and thus it is difficult to reduce the thickness of the apparatus.

### SUMMARY OF THE INVENTION

Preferred embodiments of the present invention provide biological sound measurement apparatuses that achieve reduced thicknesses, enable improvements in measurement sensitivity by increasing the internal pressures in spaces in which sound detector are housed, and enable maintaining high internal pressures over long periods of time.

A biological sound measurement apparatus according to a preferred embodiment of the present invention is a biological sound measurement apparatus that measures a biological sound of a biological body in a contact state of being in contact with a body surface of the biological body, the biological sound measurement apparatus including a sound detector; a housing that includes an opening and defines a housing space in which the sound detector is housed; and a housing cover that closes the opening from outside the housing space, defines a pressure receiving portion that receives pressure from the body surface, and covers the housing, wherein an outer wall surface of the housing include a first wall surface that protrudes farthest toward the body surface in the contact state and includes the opening provided therein, at least one step wall surface that is located nearer to the sound detector than the first wall surface is, a first side wall surface that connects a level difference between the step wall surface and the first wall surface, and a second side wall surface that is bent toward the sound detector relative to the step wall surface, and the housing cover includes the pressure receiving portion that faces the first wall surface, and a corresponding wall portion that is in close contact with the step wall surface and at least one of the first side wall surface and the second side wall surface.

According to biological sound measurement apparatuses of preferred embodiments of the present invention, it is possible to achieve reductions in thicknesses, while also enabling improvements in measurement sensitivity by increasing the internal pressures of spaces in which sound detectors are housed, and enabling maintaining high internal pressures over long periods of time.

The above and other elements, features, steps, characteristics and advantages of the present invention will become more apparent from the following detailed description of the preferred embodiments with reference to the attached drawings.

### BRIEF DESCRIPTION OF THE DRAWINGS

FIG. 1 is a side view of a biological sound measurement apparatus 1 according to a first preferred embodiment of the present invention.

FIG. 2 is a cross-sectional illustrative view taken along line A-A in the biological sound measurement apparatus 1 shown in FIG. 1.

FIG. 3 is an exploded perspective view of a detection portion is of the biological sound measurement apparatus 1 shown in FIG. 1.

FIG. 4 is a schematic cross-sectional diagram for illustrating an action during assembly of the detection portion is of the biological sound measurement apparatus 1 shown in FIG. 1.

FIG. 5 is a cross-sectional illustrative diagram showing a schematic configuration of a detection portion 10s that is a variation of the detection portion is shown in FIG. 2.

FIG. 6 is a cross-sectional illustrative diagram showing a schematic configuration of a detection portion 20s that is a variation of the detection portion is shown in FIG. 2.

FIG. 7 is a partial cross-sectional illustrative diagram showing a variation of a housing cover 4 of the detection portion is shown in FIG. 2.

FIG. 8 is an external perspective view of a biological sound measurement apparatus 2 according to a second preferred embodiment of the present invention.

FIG. 9 is a cross-sectional illustrative diagram taken along line B-B in the biological sound measurement apparatus 2 shown in FIG. 8.

FIG. 10 is a cross-sectional illustrative diagram showing a schematic configuration of a biological sound measurement apparatus 2A that is a variation of the biological sound measurement apparatus 2 shown in FIG. 9.

### DETAILED DESCRIPTION OF THE PREFERRED EMBODIMENTS

### First Preferred Embodiment

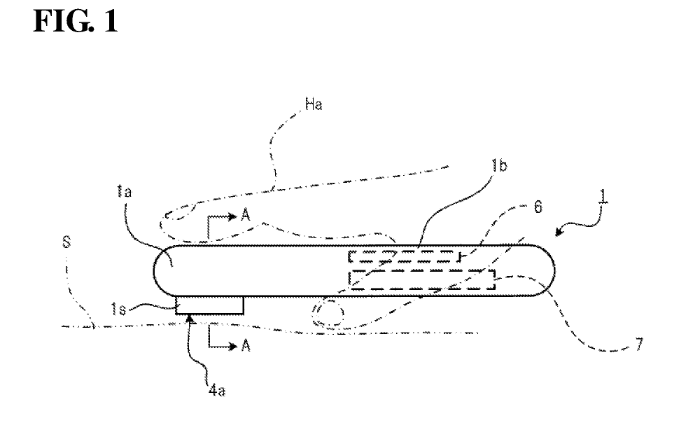

FIG. 1 is a side view of a biological sound measurement apparatus 1 according to a first preferred embodiment of the present invention.

As shown in FIG. 1, the biological sound measurement apparatus 1 includes a main body portion 1b including a housing made of a resin, a metal, or the like, and a head portion 1a is provided on one end side of the main body portion 1b.

A controller 6 that performs various types of signal processing, a battery 7 that supplies voltage necessary for operation, and the like are provided inside the main body portion 1b.

The head portion 1a is provided with a detection portion 1s that protrudes toward one side (downward in FIG. 1) in a direction that is orthogonal or substantially orthogonal to the lengthwise direction of the biological sound measurement apparatus 1. A pressure receiving portion 4a, which is brought into contact with a body surface S of a biological body that is the subject, and which receives pressure from the body surface S, is provided at the leading end of the detection portion 1s.

The biological sound measurement apparatus 1 preferably has a size that enables being gripped and operated by an adult hand, for example.

The biological sound measurement apparatus 1 is used in a state where the pointer finger or the like of a hand Ha of a user is placed on the back surface of the detection portion 1s of the main body portion 1b, and the pressure receiving portion 4a of the detection portion 1s is pressed against the body surface S by the pointer finger. Hereinafter, the direction in which the pressure receiving portion 4a is pressed against the body surface S will be referred to as the pressing direction (direction from the top to the bottom in FIG. 1).

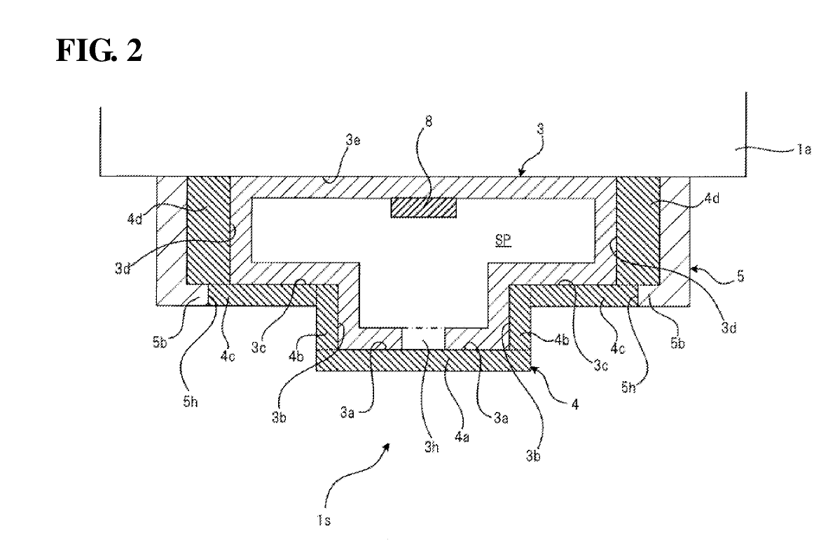

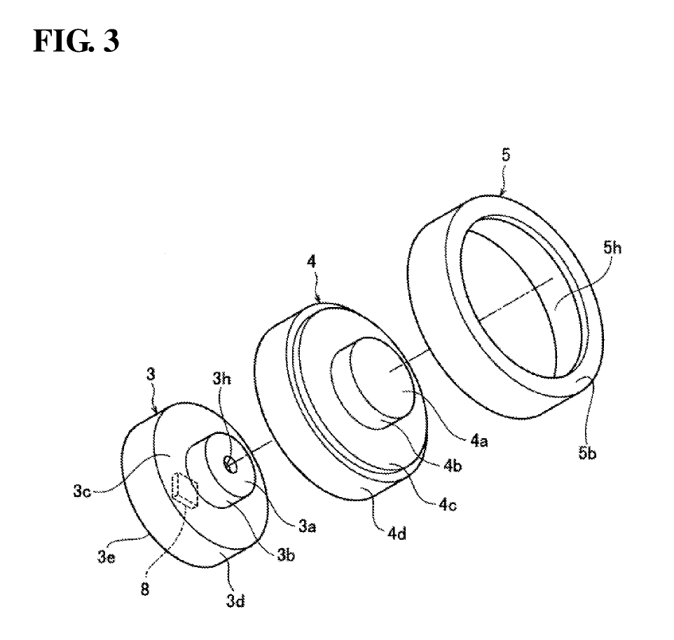

FIG. 2 is a cross-sectional illustrative view taken along line A-A in the biological sound measurement apparatus 1 shown in FIG. 1. FIG. 3 is an exploded perspective view of the detection portion 1s of the biological sound measurement apparatus 1 shown in FIG. 1.

The detection portion 1s includes a sound detector 8 that detects sound and includes a MEMS (Micro Electro Mechanical Systems) microphone or a capacitance microphone or the like, a housing 3 that includes an opening 3h and defines a housing space SP in which the sound detector 8 is housed, a housing cover 4 that closes the opening 3h from outside the housing space SP and covers the housing 3, and an outer cover 5 that covers the housing cover 4 while exposing a portion of the housing cover 4.

The sound detector 8 is electrically connected to the controller 6 shown in FIG. 1 by a lead line (not shown) or the like, and transmits information regarding a detected biological sound to the controller 6.

The housing 3 protrudes along the pressing direction, and is made of a material that has a high rigidity and has a higher acoustic impedance than air, such as a resin or a metal.

The outer wall surface of the housing 3 includes a bent structure including a ring-shaped first wall surface 3a that protrudes the farthest toward the body surface S in a contact state where the pressure receiving portion 4a is in contact with the body surface S, is perpendicular to the pressing direction, and is the surface in which the opening 3h is provided; a circular or substantially circular bottom wall surface 3e that is parallel or substantially parallel with the first wall surface 3a, and is located at a position farthest from the first wall surface 3a; a ring-shaped step wall surface 3c that is parallel or substantially parallel with the first wall surface 3a, and is located on the side opposite to the body surface S side relative to the first wall surface 3a in the aforementioned contact state (i.e., is located nearer to the sound detector 8 in the pressing direction than the first wall surface 3a is); a first side wall surface 3b that connects a level difference between the inner peripheral edge of the step wall surface 3c and the outer peripheral edge of the first wall surface; and a second side wall surface 3d that connects a level difference between the outer peripheral edge of the step wall surface 3c and the outer peripheral edge of the circular bottom wall surface 3e, and is bent to the sound detector 8 side (direction opposite to the pressing direction) relative to the step wall surface 3c.

In a view along the pressing direction, the outer peripheral edge of the first wall surface 3a and the inner peripheral edge of the step wall surface 3c overlap each other. Accordingly, the first side wall surface 3b is a bent surface that is equivalent to the side surface of a circular column defined by a locus when the outer peripheral edge of the first wall surface 3a is moved in the pressing direction.

In a view along the pressing direction, the outer peripheral edge of the step wall surface 3c and the outer peripheral edge of the bottom wall surface 3e are overlapped with each other. Accordingly, the second side wall surface 3d is a bent surface that is equivalent to the side surface of a circular column defined by a locus when the outer peripheral edge of the step wall surface 3c is moved in the pressing direction.

Note that in a view along the pressing direction, the inner peripheral edge of the step wall surface 3c may be larger than the outer peripheral edge of the first wall surface 3a. In this case, the first side wall surface 3b is a bent surface that is equivalent to the side surface of a three-dimensional object obtained by cutting the apex of a cone.

Also, in a view along the pressing direction, the outer peripheral edge of the bottom wall surface 3e may be larger than the outer peripheral edge of the step wall surface 3c. In this case, the second side wall surface 3d is a bent surface that is equivalent to the side surface of a three-dimensional object obtained by cutting the apex of a cone.

The housing 3 having the above-described configuration is supported by a housing that defines the head portion 1a. Specifically, the bottom wall surface 3e of the housing 3 is fixed to the housing that defines the head portion 1a by a screw fastening mechanism, an adhesive, or the like.

The housing cover 4 is a bottomed tube-shaped member, and the shape of the hollow portion thereof substantially matches the outer wall shape of the housing 3.

Specifically, the housing cover 4 is define by a flat plate-shaped pressure receiving portion 4a that closes the opening 3h provided in the first wall surface 3a of the housing 3, maintains the air-tight state of the housing space SP, and is in close contact with the first wall surface 3a; a circular tube-shaped first corresponding wall portion 4b that is in close contact with the first side wall surface 3b of the housing 3; a circular tube-shaped second corresponding wall portion 4c that is in close contact with the step wall surface 3c of the housing 3; and a circular tube-shaped third corresponding wall portion 4d that is in close contact with the second side wall surface 3d of the housing 3. The third corresponding wall portion 4d has a larger outer diameter than the second corresponding wall portion 4c.

Note that in order to describe the wall portions that define the housing cover 4, the wall portions are shown with use of dashed lines in the housing cover 4 in FIG. 2, but in actuality, the housing cover 4 is defined by integral molding of a material in a metal mold or the like.

The housing cover 4 is defined by a material that has an acoustic impedance close to that of a human body, air, or water, and that is flexible and has favorable biocompatibility. Examples of the material of the housing cover 4 include silicone and an elastomer. The thickness of the housing cover 4 preferably is in the range of about 0.3 mm to about 1.0 mm inclusive, for example, but there is no limitation to this.

When the biological sound measurement apparatus 1 is used, the pressure receiving portion 4a of the housing cover 4 is brought into contact with the body surface S. When the pressure receiving portion 4a vibrates due to a biological sound, the internal pressure of the housing space SP fluctuates due to the vibration, and, due to this internal pressure fluctuation, an electrical signal that corresponds to the biological sound is detected by the sound detector 8.

The controller 6 shown in FIG. 1 processes information regarding the biological sound detected by the sound detector 8, makes a determination regarding the presence/absence of wheezing, a determination regarding a cardiac sound abnormality, or the like, and informs the user of the determination result by audio, display, or the like.

Note that the housing cover 4 and the housing 3 are put in a close contact state by being directly brought into contact with each other without a substance such as an adhesive therebetween, and thus the housing cover 4 is able to be attached to and removed from the housing 3. Note that the detection portion 1s may have a configuration in which the first corresponding wall portion 4b and the first side wall surface 3b are not in close contact, or a configuration in which the third corresponding wall portion 4d and the second side wall surface 3d are not in close contact.

The outer cover 5 is a member that covers the housing cover 4 in a state where the pressure receiving portion 4a of the housing cover 4 is exposed. The outer cover 5 is a tube-shaped member that is structured to be able to be attached to and removed from the head portion 1a by snap-fitting or the like.

In the state of being attached to the head portion 1a as shown in FIG. 2, the outer cover 5 includes a pressing wall portion 5b that engages with a level difference portion between the second corresponding wall portion 4c and the third corresponding wall portion 4d of the housing cover 4 and presses a portion of the housing cover 4 toward the head portion 1a, and a pressure receiving portion opening 5h that exposes the pressure receiving portion 4a of the housing cover 4.

The outer cover 5 is preferably defined by a lightweight material such as ABS resin in order to enable pressing the flexible housing cover 4 toward the head portion 1a with an appropriate amount of pressure. Also, the outer cover 5 is defined by a material that has a higher rigidity than the housing cover 4.

In the example shown in FIG. 2, the outer surface of the pressing wall portion 5b of the outer cover 5 is located nearer to the head portion 1a than the outer surface of the pressure receiving portion 4a of the housing cover 4. Accordingly, even in the state where the pressure receiving portion 4a is in contact with the body surface S, the outer cover 5 is prevented from coming into contact with body surface S.

As shown in FIG. 3, the detection portion 1s having the above-described configuration can be assembled by a process in which the three-level circular tube-shaped housing cover 4 is placed over the housing 3 from above the first wall surface 3a, and then the circular tube-shaped outer cover 5 having the pressure receiving portion opening 5h is placed on the housing cover 4.

No connection means (e.g., an adhesive) whatsoever is used between the members that are fitted together in this way, and the members are locked by friction engagement achieved by the state of pressure contact between the members.

Specifically, the second corresponding wall portion 4c of the housing cover 4 is in pressure contact with the step wall surface 3c of the housing 3, the first corresponding wall portion 4b of the housing cover 4 is in pressure contact with the first side wall surface 3b of the housing 3, the pressure receiving portion 4a of the housing cover 4 is in pressure contact with the first wall surface 3a of the housing 3, and the third corresponding wall portion 4d of the housing cover 4 is in pressure contact with the second side wall surface 3d of the housing 3.

Furthermore, the outer cover 5 is in pressure contact with the third corresponding wall portion 4d of the housing cover 4. In this way, three members are fitted together in close contact, and thus these members are fitted together in an air-tight state.

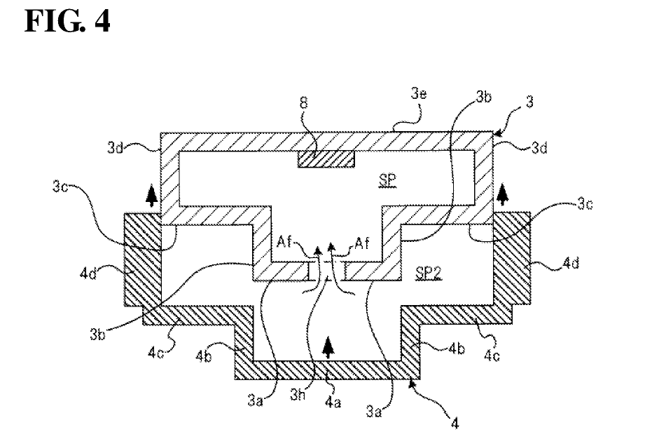

FIG. 4 is a schematic cross-sectional diagram for illustrating an action during assembly of the detection portion is of the biological sound measurement apparatus 1 shown in FIG. 1.

As shown in FIG. 4, in an initial stage where the housing cover 4 is placed over the housing 3, the housing space SP is provided inside the housing 3, and a space SP2 is defined by the housing 3 and the housing cover 4.

The housing cover 4 in the state shown in FIG. 4 is then pressed toward the housing 3. When this pressing operation is performed, the housing cover 4 moves (move in the direction indicated by the large arrow in the figure) in a state where the second side wall surface 3d of the housing 3 and the third corresponding wall portion 4d of the housing cover 4 are in close contact.

Accordingly, the air inside the space SP2 is pressed from the opening 3h toward the housing space SP as inflow air Af, with almost no leakage to the outside. Ultimately, all of the air in the space SP2 is pressed into the housing space SP. As a result, the internal pressure of the housing space SP is set to a high pressure state.

The outer cover 5 is then fitted to the housing cover 4, and thus the mated state of the housing 3 and the housing cover 4 is fixed even more reliably, and the high pressure inside the housing space SP is maintained with almost no leakage of the air inside the housing space SP. In this way, the pressure inside the housing space SP is in a high state, thus increasing the precision of transmission of vibration from the pressure receiving portion 4a to the sound detector 8.

As described above, the biological sound measurement apparatus 1 has a configuration in which the outer wall surface of the housing 3 includes the first wall surface 3a and a bent surface that is bent three or more times relative to the first wall surface 3a (i.e., the first side wall surface 3b, the step wall surface 3c, and the second side wall surface 3d), and the housing cover 4 is in close contact with the first wall surface 3a and this bent surface.

For this reason, in comparison with a conventional configuration, it is possible to increase the area of close contact between the housing cover 4 and the housing 3, and it is possible to increase the air-tightness between the housing 3 and the housing cover 4.

Also, the housing 3 and the housing cover 4 both have a bent structure, and therefore the air in the space SP2 is not likely to leak when these two members are fitted together, thus making it possible to increase the internal pressure of the housing space SP.

Also, due to the housing 3 and the housing cover 4 both having a bent structure, the rigidity of these two members increases, it is possible to increase the contact pressure when fitting these two members together, and it is possible to increase the degree of close contact between these two members.

In this way, according to the biological sound measurement apparatus 1, it is possible to increase the internal pressure of the housing space SP when assembling the detection portion is, and it is possible to improve the precision of biological sound detection by the sound detector 8.

Also, by increasing the degree of close contact between the housing 3 and the housing cover 4, it is possible to maintain the internal pressure of the housing space SP over a long period of time after assembly of the detection portion is as well. Accordingly, it is possible to maintain the improvement in biological sound measurement precision over a long period of time.

Also, by increasing the degree of close contact between the housing 3 and the housing cover 4, it is possible to suppress shifting of the housing cover 4 caused by rubbing between the pressure receiving portion 4a and the body surface S, and it is possible to prevent a decrease in biological sound measurement precision.

Also, due to the housing 3 and the housing cover 4 having a bent structure, it is possible to increase the degree of close contact between the housing 3 and the housing cover 4 without increasing the thickness thereof. Also, even if the width of the housing 3 in the pressing direction is reduced, it is possible to ensure close contact between the housing 3 and the housing cover 4, thus making it possible to realize a reduction in the thickness of the detection portion 1s.

Also, according to the biological sound measurement apparatus 1, the housing cover 4 can be attached to and removed from the housing 3. For this reason, even if the housing cover 4 becomes soiled or damaged due to extended use, the apparatus is able to continue to be used by replacing the housing cover 4.

Also, even if the internal pressure of the housing space SP decreases due to extended use or some other factor, by removing the housing cover 4 from the housing 3 and then reattaching it, it is possible to restore the internal pressure of the housing space SP to a high state, and it is possible to improve the biological sound measurement precision.

Moreover, according to the biological sound measurement apparatus 1, the outer cover 5 that presses a portion of the housing cover 4 toward the head portion 1a is provided, thus further improving the degree of close contact between the housing 3 and the housing cover 4.

As a result, the air-tightness of the housing space SP is maintained more reliably, and it is possible to improve the ability to maintain the internal pressure of the housing space SP. Also, the outer cover 5 makes it possible to improve the effect of suppressing shifting of the housing cover 4 caused by rubbing between the body surface S and the pressure receiving portion 4a.

Furthermore, the biological sound measurement apparatus 1 has a configuration in which the outer surface of the pressing wall portion 5b of the outer cover 5 is located nearer to the sound detector 8 than the outer surface of the pressure receiving portion 4a, and the pressing wall portion 5b is not likely to come into contact with the body surface S when the apparatus is used.

For this reason, the material of the outer cover 5 can be selected without giving consideration to contact with the body surface S, and can be a material that is specialized for the function of holding the housing cover 4, thus making it possible to reduce the apparatus manufacturing cost.

Note that a configuration is possible in which the shape of the hollow portion of the housing cover 4 is preferably slightly smaller than the shape of the outer wall surface of the housing 3, and the housing cover 4 and the housing 3 are fitted together by pressing the housing 3 into the hollow portion of the housing cover 4. According to this configuration, it is possible to omit the outer cover 5, and it is possible to reduce the apparatus manufacturing cost.

### First Variation

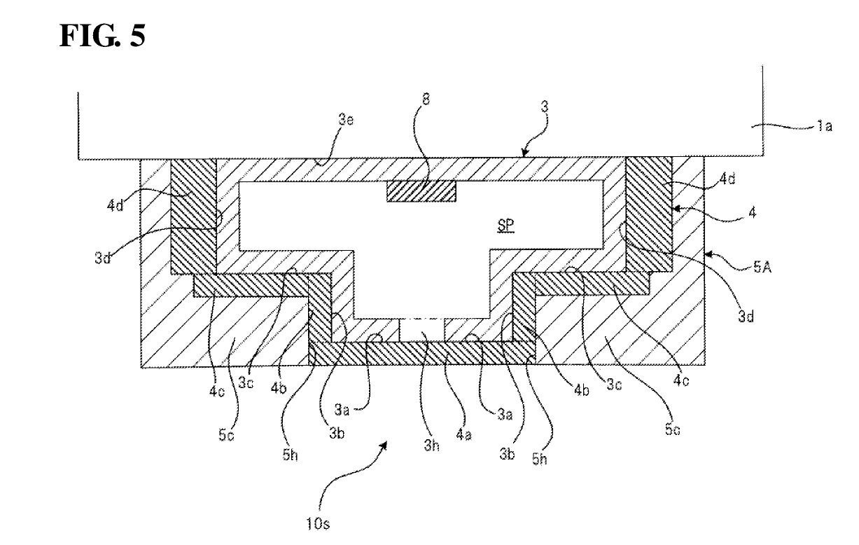

FIG. 5 is a cross-sectional illustrative diagram showing a schematic configuration of a detection portion 10s that is a variation of the detection portion is shown in FIG. 2. The detection portion 10s preferably has the same configuration as the detection portion is, with the exception that the outer cover 5 has been changed to an outer cover 5A. The outer cover 5A will be described below.

The outer cover 5A is a tube-shaped member that is structured to be able to be attached to and removed from the head portion 1a by snap-fitting or the like, and the shape of the hollow portion substantially matches the shape of the outer surface of the housing cover 4.

In the state of being attached to the head portion 1a as shown in FIG. 5, the outer cover 5A includes a step portion between the second corresponding wall portion 4c and the third corresponding wall portion 4d, a pressing wall portion 5c that is engaged with the step portion between the second corresponding wall portion 4c and the first corresponding wall portion 4b and presses a portion of the housing cover 4 toward the head portion la, and a pressure receiving portion opening 5h that exposes the pressure receiving portion 4a of the housing cover 4.

In the example shown in FIG. 5, the outer surface of the pressing wall portion 5c of the outer cover 5A is located on the same or substantially the same plane as the outer surface of the pressure receiving portion 4a of the housing cover 4. Note that the term “substantially the same plane” does not mean a complete match, and allows for a certain degree of dimensional error in manufacturing, and thus a certain degree of shift is allowed.

According to the detection portion 10s having the above-described configuration, the outer surface of the outer cover 5A is on the same or substantially the same plane as the outer surface of the pressure receiving portion 4a, and therefore it is possible to increase the area of contact between the detection portion 10s and the body surface S.

For this reason, it is possible to reduce the pressure with which the pressure receiving portion 4a is brought into contact with the body surface S, and mitigate the burden on the subject from this contact pressure. Also, there are no level differences between the outer cover 5A and the pressure receiving portion 4a, contact of the detection portion 10s with the body surface S is favorable, and it is possible to improve the usability of the apparatus.

### Second Variation

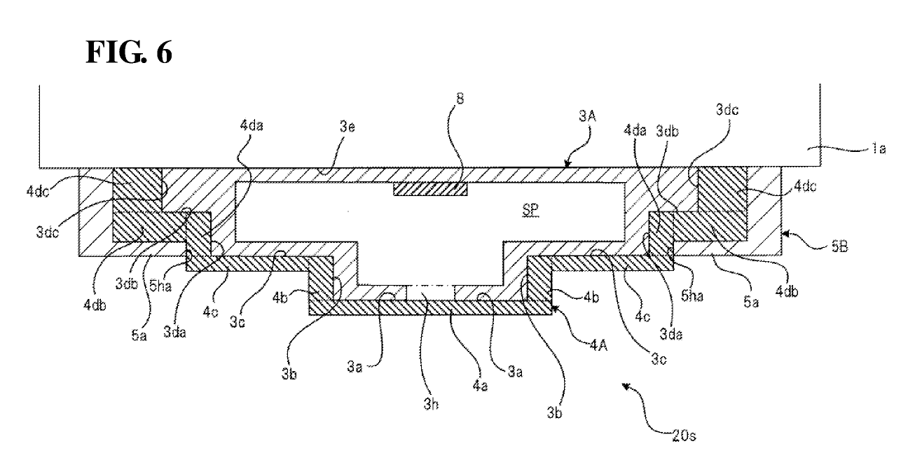

FIG. 6 is a cross-sectional illustrative diagram showing a schematic configuration of a detection portion 20s that is a variation of the detection portion is shown in FIG. 2.

The detection portion 20s shown in FIG. 6 preferably has the same configuration as the detection portion is in FIG. 2, with the exceptions that the housing 3 has been changed to a housing 3A, the housing cover 4 has been changed to a housing cover 4A, and the outer cover 5 has been changed to an outer cover 5B.

The housing 3A has a configuration in which the second side wall surface 3d of the housing 3 shown in FIG. 2 has been changed to a bent surface that is defined by a second side wall surface 3da, a step wall surface 3db, and a third side wall surface 3dc.

The step wall surface 3db is a ring-shaped flat face that is parallel or substantially parallel with the step wall surface 3c and is located on the side opposite to the body surface S side relative to the step wall surface 3c in the contact state where the pressure receiving portion 4a is in contact with the body surface S.

The second side wall surface 3da is a bent surface that connects a level difference between the step wall surface 3c and the step wall surface 3db.

The third side wall surface 3dc is a bent surface that is bent to the sound detector 8 side (direction opposite to the pressing direction) relative to the step wall surface 3db, and connects a level difference between the step wall surface 3db and the bottom wall surface 3e.

In this way, the outer wall surface of the housing 3A includes the first wall surface 3a and a bent surface that is bent five times relative to the first wall surface 3a (i.e., the first side wall surface 3b, the step wall surface 3c, the second side wall surface 3da, the step wall surface 3db, and the third side wall surface 3dc).

The housing cover 4A has a configuration in which the third corresponding wall portion 4d of the housing cover 4 shown in FIG. 2 has been changed to a bent structure that is defined by a third corresponding wall portion 4da, a fourth corresponding wall portion 4db, and a fifth corresponding wall portion 4dc.

The third corresponding wall portion 4da is a tube-shaped portion that is in close contact with the second side wall surface 3da of the housing 3A.

The fourth corresponding wall portion 4db is a tube-shaped portion that is in close contact with the step wall surface 3db of the housing 3A.

The fifth corresponding wall portion 4dc is a tube-shaped portion that is in close contact with the third side wall surface 3dc of the housing 3A.

Note that in order to describe the wall portions of the housing cover 4A, the wall portions are shown with use of dashed lines in the housing cover 4A in FIG. 6, but in actuality, the housing cover 4A is formed preferably by integral molding of a material in a metal mold or the like.

The outer cover 5B has been changed to a configuration including a pressing wall portion 5a that is engaged with a level difference portion between the third corresponding wall portion 4da and the fourth corresponding wall portion 4db and presses a portion of the housing cover 4A toward the head portion 1a, and a pressure receiving portion opening 5ha that exposes the pressure receiving portion 4a of the housing cover 4A.

As shown in FIG. 6, the outer wall surface of the housing 3A is provided with the two step wall surfaces 3c and 3db, the first side wall surface 3b, the second side wall surface 3da, and the third side wall surface 3dc, and thus the housing cover 4A is also provided with five corresponding wall portions that correspond to this structure. Therefore, according to the detection portion 20s, it is possible to further improve the degree of close contact between the housing 3A and the housing cover 4A, and it is possible to improve the biological sound measurement precision. Also, it is possible to further reduce the thickness of the housing 3A, and apparatus size reduction and weight reduction are able to be achieved.

Note that the detection portion 20s shown in FIG. 6 may have a configuration in which the first corresponding wall portion 4b and the first side wall surface 3b are not in close contact, a configuration in which the third corresponding wall portion 4da and the second side wall surface 3da are not in close contact, or a configuration in which the fifth corresponding wall portion 4dc and the third side wall surface 3dc are not in close contact. Also, the detection portion 20s shown in FIG. 6 may have a configuration in which the first corresponding wall portion 4b and the first side wall surface 3b are not in close contact, and furthermore the third corresponding wall portion 4da and the second side wall surface 3da are not in close contact. Also, the detection portion 20s shown in FIG. 6 may have a configuration in which the third corresponding wall portion 4da and the second side wall surface 3da are not in close contact, and furthermore the fifth corresponding wall portion 4dc and the third side wall surface 3dc are not in close contact.

### Third Variation

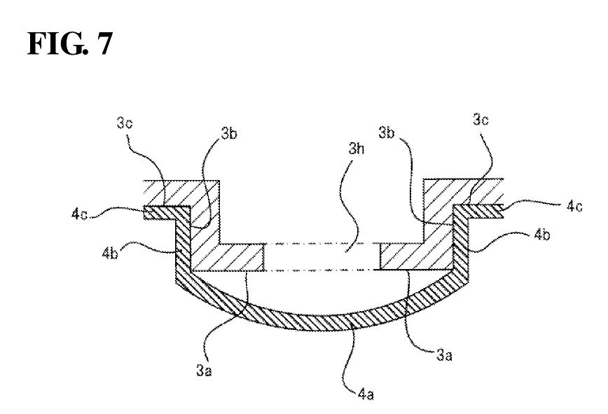

FIG. 7 is a partial cross-sectional illustrative diagram showing a variation of the housing cover 4 of the detection portion is shown in FIG. 2. FIG. 7 is an enlarged view of the variation in a region including the pressure receiving portion 4a of the housing cover 4 shown in FIG. 2.

The pressure receiving portion 4a of the variation shown in FIG. 7 has a curved shape that bulges in a direction of separation from the housing 3. In a state of not being in contact with the body surface S, the pressure receiving portion 4a bulges as shown in FIG. 7 due the internal pressure of the housing space SP, and maintains this shape while facing the first wall surface 3a. On the other hand, when brought into contact with the body surface S, the pressure receiving portion 4a freely deforms so as to conform to the shape of the body surface S.

Accordingly, it is possible to improve the ability of the pressure receiving portion 4a to conform to the body surface S, and even a user who is not accustomed to operating the apparatus can easily obtain a precise contact state, and it is possible to improve the biological sound measurement precision.

Note that the configuration of the pressure receiving portion 4a shown in FIG. 7 can be similarly applied to the detection portions 10s and 20s shown in FIGS. 5 and 6 as well.

### Second Preferred Embodiment

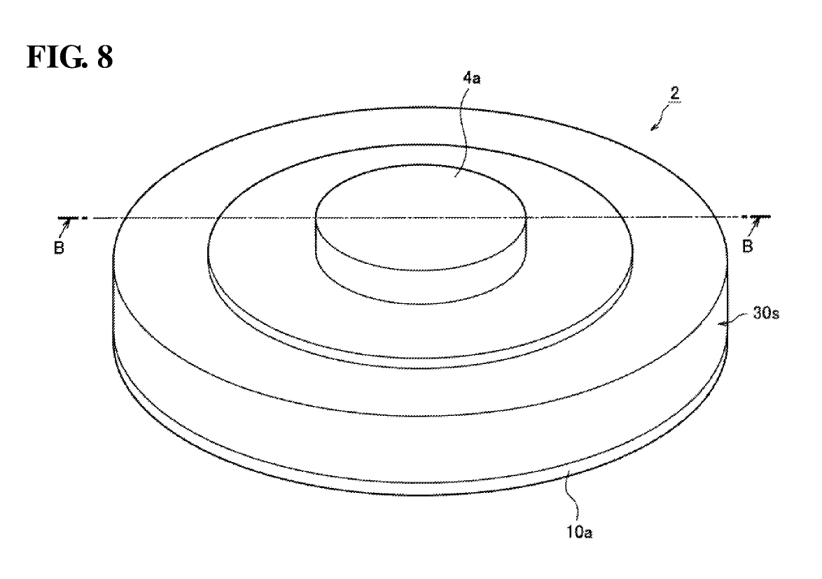

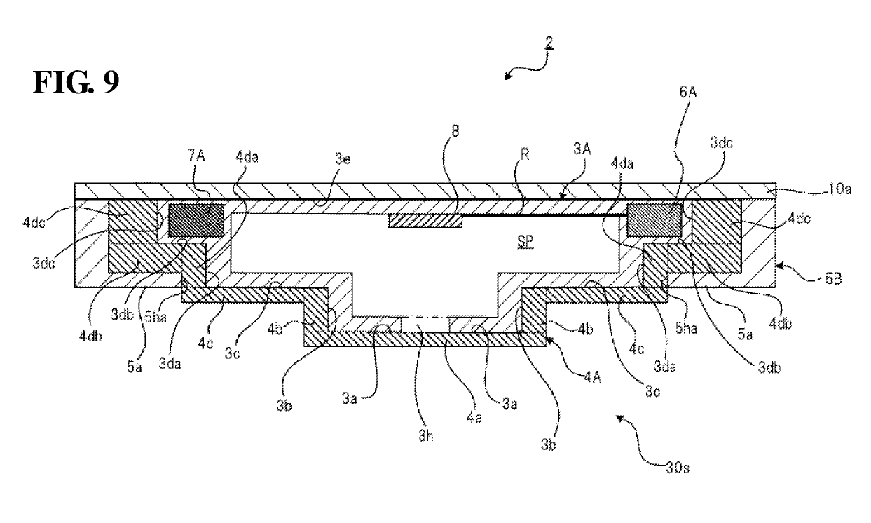

FIG. 8 is an external perspective view of a biological sound measurement apparatus 2 according to a second preferred embodiment of the present invention. FIG. 9 is a cross-sectional illustrative diagram taken along line B-B in the biological sound measurement apparatus 2 shown in FIG. 8.

The biological sound measurement apparatus 2 is not a handheld type of apparatus that a user grips with a hand, but rather is a type of apparatus that is affixed to a body surface of a biological body and used over a long period of time. As shown in FIG. 8, the biological sound measurement apparatus 2 is defined by a detection portion 30s that includes a pressure receiving portion 4a, and a support member 10a that supports the detection portion 30s.

The support member 10a is defined by a lightweight material such as ABS resin, in consideration of the fact that the biological sound measurement apparatus 2 is attached to the body surface S for a long period of time.

The detection portion 30s shown in FIG. 9 preferably has the same configuration as the detection portion 20s shown in FIG. 6, with the exception that a circuit substrate 6A and a compact battery 7A such as a coin cell are housed inside the housing 3A.

The housing 3A of the detection portion 30s is supported by the support member 10a by being fixed to the support member 10a with use of a screw fastening mechanism, an adhesive, or the like. Also, the outer cover 5B of the detection portion 30s can be attached to and removed from the support member 10a by a screw fastening mechanism, snap-fitting, or the like.

The sound detector 8 of the detection portion 30s and the circuit substrate 6A are connected by a lead line R. Note that in order to ensure the air-tightness of the housing space SP, a gap in a portion of the housing 3A through which the lead line R passes is filled with a shrinkable rubber member or the like.

A circuit that operates using voltage supplied from the battery 7A is implemented on the circuit substrate 6A, and this circuit includes a communication module that acquires information regarding biological sound detected by the sound detector 8 and transfers the acquired information to an external device such as a personal computer or a smartphone. This circuit may include a circuit that has the same functions as the controller 6 described above. A module that is compliant with a short-range wireless communication standard such as Bluetooth (registered trademark) is preferably used as the communication module, for example.

The biological sound measurement apparatus 2 is used in a state where medical double-sided tape is affixed to the pressure receiving portion 4a, and furthermore affixed to the body surface S of a biological body. Also, the biological sound measurement apparatus 2 is used in a state of being affixed to the body surface S by medical tape that covers the entirety of the biological sound measurement apparatus 2 from above the support member 10a. Tape that is defined by a polyethylene core, a polyester core, a rayon nonwoven cloth, or the like can be used as the medical tape.

Note that the biological sound measurement apparatus 2 is used in a state where the pressure receiving portion 4a of the biological sound measurement apparatus 2 is pressed against the body surface S by the above-described tape. For this reason, in FIG. 9, the pressing direction in which the pressure receiving portion 4a is pressed against the body surface S is the direction from the top to the bottom in the figure, similarly to the first preferred embodiment.

The detection portion 30s of the biological sound measurement apparatus 2 has a configuration similar to that of the detection portion 20s, and therefore there is a high degree of close contact between the housing 3A and the housing cover 4A, and it is possible to ensure biological sound detection precision even if the height of the housing 3A is reduced.

Accordingly, it is possible to reduce the thickness and weight of the biological sound measurement apparatus 2, thus making it possible to mitigate the burden on the subject even when used for a long period of time. Also, by using flexible members for the circuit substrate 6A and the battery 7A, it is possible to achieve a further reduction in thickness and size.

Moreover, according to the biological sound measurement apparatus 2, it is possible to replace the housing cover 4A, thus making it possible to mitigate concerns regarding sanitation when used with a large number of people or used for a long period of time.

Note that instead of the detection portion 30s of the biological sound measurement apparatus 2, a configuration is possible in which the detection portion is in FIG. 2 or the detection portion 10s in FIG. 5 is used, and the circuit substrate 6A and the battery 7A are provided inside the housing 3 of the detection portion is or the detection portion 10s. With this configuration as well, it is possible to provide a compact and thin biological sound measurement apparatus that is suited to continuous use.

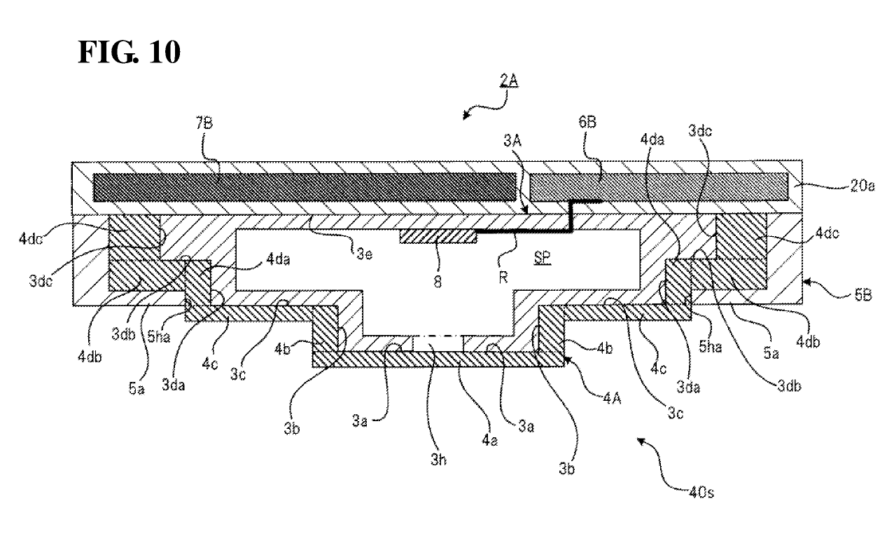

FIG. 10 is a cross-sectional illustrative diagram showing a schematic configuration of a biological sound measurement apparatus 2A that is a variation of the biological sound measurement apparatus 2 shown in FIG. 9.

The biological sound measurement apparatus 2A is different from the biological sound measurement apparatus 2 in that the detection portion 30s has been changed to a detection portion 40s, and the support member 10a has been changed to a support member 20a.

The detection portion 40s preferably has the same configuration as the detection portion 20s shown in FIG. 6.

The support member 20a is a member that supports the detection portion 40s, and is defined by ABS resin or the like. Also, a circuit substrate 6B and a compact battery 7B such as a coin cell are provided inside the support member 20a.

The housing 3A of the detection portion 40s is supported by the support member 20a by being fixed to the support member 20a with use of a screw fastening mechanism, an adhesive, or the like. Also, the outer cover 5B of the detection portion 40s can be attached to and removed from the support member 20a by a screw fastening mechanism, snap-fitting, or the like.

The sound detector 8 of the detection portion 40s and the circuit substrate 6B are connected by a lead line R that passes through the housing 3A and the support member 20a. Note that in order to ensure the air-tightness of the housing space SP, a gap in a portion of the housing 3A through which the lead line R passes is filled with a shrinkable rubber member or the like.

A circuit that operates using voltage supplied from the battery 7B is implemented on the circuit substrate 6B, and the circuit substrate 6B preferably has the same configuration as the circuit substrate 6A.

According to the biological sound measurement apparatus 2A having this configuration, it is possible to obtain effects similar to those of the biological sound measurement apparatus 2.

Note that in the biological sound measurement apparatuses 2 and 2A as well, a configuration is possible in which the shape of the hollow portion of the housing cover 4A is set slightly smaller than the outer shape of the housing 3A, and the housing cover 4A and the housing 3A are fitted together by pressing the housing 3A into the hollow portion of the housing cover 4A. According to this configuration, it is possible to omit the outer cover 5B, and it is possible to reduce the apparatus manufacturing cost and achieve a further reduction in size and weight.

Although preferred embodiments of the present invention have been described above, the present invention is not limited to these preferred embodiments, and can be changed as appropriate. For example, although the housing and the housing cover of the detection portion is preferably have a circular planar shape, the shapes are not limited to being circular, and may be elliptical or approximately rectangular. Also, although the step wall surfaces 3c and 3db are flat surfaces that are perpendicular or substantially perpendicular to the pressing direction, they may be inclined bent surfaces that are not perpendicular to the pressing direction.

As described above, the present specification discloses the following matter.

A biological sound measurement apparatus according to a preferred embodiment of the present invention is a biological sound measurement apparatus that measures a biological sound of a biological body in a contact state of being in contact with a body surface of the biological body, the biological sound measurement apparatus including a sound detector; a housing that includes an opening and defines a housing space in which the sound detector is housed; and a housing cover that closes the opening from outside the housing space, defines a pressure receiving portion that receives pressure from the body surface, and covers the housing, wherein an outer wall surface of the housing includes a first wall surface that protrudes farthest toward the body surface in the contact state and includes the opening provided therein, at least one step wall surface that is located nearer to the sound detector than the first wall surface is, a first side wall surface that connects a level difference between the step wall surface and the first wall surface, and a second side wall surface that is bent toward the sound detector relative to the step wall surface, and the housing cover includes the pressure receiving portion that faces the first wall surface, and a corresponding wall portion that is in close contact with the step wall surface and at least one of the first side wall surface and the second side wall surface.

A biological sound measurement apparatus according to a preferred embodiment of the present invention also includes a support member that supports the housing; and an outer cover that includes a pressing wall portion that presses a portion of the housing cover toward the support member, and covers the housing cover in a state where the pressure receiving portion is exposed.

According to a biological sound measurement apparatus according to a preferred embodiment of the present invention, an outer surface of the pressing wall portion is located on the same or substantially the same plane as an outer surface of the pressure receiving portion.

According to a biological sound measurement apparatus according to a preferred embodiment of the present invention, an outer surface of the pressing wall portion is located nearer to the support member than an outer surface of the pressure receiving portion.

According to a biological sound measurement apparatus according to a preferred embodiment of the present invention, an outer wall of the housing includes the first wall surface, two of the step wall surfaces, the first side wall surface that connects a level difference between the first wall surface and one of the two step wall surfaces, the second side wall surface that connects a level difference between the two step wall surfaces, and a third side wall surface that is bent toward the sound detector relative to another one of the two step wall surfaces, and the housing cover includes a corresponding wall portion that is in close contact with each of the two step wall surfaces and at least one of the first side wall surface, the second side wall surface, and the third side wall surface.

According to a biological sound measurement apparatus according to a preferred embodiment of the present invention, the pressure receiving portion has a curved shape that bulges in a direction of separation from the housing.

According to a biological sound measurement apparatus according to a preferred embodiment of the present invention, the housing cover is structured to be able to be attached to and removed from the housing.

While preferred embodiments of the present invention have been described above, it is to be understood that variations and modifications will be apparent to those skilled in the art without departing from the scope and spirit of the present invention. The scope of the present invention, therefore, is to be determined solely by the following claims.

What is claimed is:

1. A biological sound measurement apparatus that measures a biological sound of a biological body in a contact state of being in contact with a body surface of the biological body, the biological sound measurement apparatus comprising:
a sound detector;
a housing that includes an opening and defines a housing space in which the sound detector is housed; and
a housing cover that closes the opening from outside the housing space, defines a pressure receiving portion that receives pressure from the body surface, and covers the housing; wherein
an outer wall surface of the housing includes a first wall surface that protrudes farthest toward the body surface in the contact state and includes the opening provided therein, at least one step wall surface that is located nearer to the sound detector than the first wall surface, a first side wall surface that connects a level difference between the step wall surface and the first wall surface, and a second side wall surface that is bent toward the sound detector relative to the step wall surface; and
the housing cover includes the pressure receiving portion that faces the first wall surface, and a corresponding wall portion that is in close contact with the step wall surface and at least one of the first side wall surface and the second side wall surface.

2. The biological sound measurement apparatus according to claim 1, further comprising:
a support member that supports the housing; and
an outer cover that includes a pressing wall portion that presses a portion of the housing cover toward the support member, and covers the housing cover in a state where the pressure receiving portion is exposed.

3. The biological sound measurement apparatus according to claim 2, wherein an outer surface of the pressing wall portion is located on a same plane or substantially a same plane as an outer surface of the pressure receiving portion.

4. The biological sound measurement apparatus according to claim 2, wherein an outer surface of the pressing wall portion is closer to the support member than an outer surface of the pressure receiving portion.

5. The biological sound measurement apparatus according to claim 1, wherein
an outer wall of the housing includes the first wall surface, two of the step wall surfaces, the first side wall surface that connects a level difference between the first wall surface and one of the two step wall surfaces, the second side wall surface that connects a level difference between the two step wall surfaces, and a third side wall surface that is bent toward the sound detector relative to another one of the two step wall surfaces; and
the housing cover includes a corresponding wall portion that is in close contact with each of the two step wall surfaces and at least one of the first side wall surface, the second side wall surface, and the third side wall surface.

6. The biological sound measurement apparatus according to claim 1, wherein the pressure receiving portion has a curved shape that bulges in a direction of separation from the housing.

7. The biological sound measurement apparatus according to claim 1, wherein the housing cover is structured to be able to be attached to and removed from the housing.
